(19) United States
(12) Patent Application Publication
(10) Pub. No.: US 2022/0040458 A1
(43) Pub. Date: Feb. 10, 2022

(54) MECHANIZED SURGICAL DRAIN AND METHOD OF USE

(71) Applicant: Kenneth R. STUEBE, Alachua, FL (US)

(72) Inventor: Kenneth R. STUEBE, Alachua, FL (US)

(21) Appl. No.: 16/985,260

(22) Filed: Aug. 5, 2020

(51) Int. Cl. A61M 27/00

(52) U.S. Cl. CPC A61M 27/00; A61M 1/0001

(57) ABSTRACT

A drainage system that includes a mechanized surgical drain and drive mechanism for draining body matter from a body cavity. The mechanized surgical drain comprises a carrier mechanism within a surgical drain tube. The drive mechanism provides motion capabilities to a carrier mechanism within the lumen of the surgical drain tube. The motion capabilities of the carrier mechanism inhibits occlusion of the surgical drain tube by dislodging body matter and moving the body matter through the surgical drain to a suction port for removal to a collection container.

Chest tubes and other types of surgical drain catheters are routinely used in patients who have had cardiothoracic surgery or chest trauma to drain blood and other biological body matter from the pleural can pericardial cavities. Chest tubes help avoid complications related to accumulation of blood and thrombi (blood clots), air, debris, or other fluids in the pericardial sac and pleural space and maintain cardiorespiratory/hemodynamic stability.

It is not uncommon for chest tubes, and other drainage tubes, to become occluded with clots and solidified body matter, which can block the flow of material through the drain catheter. Such occlusions can lead to life threatening complications, such as hemothorax, acute tamponade, and pericardial effusion. Those complications can compromise postsurgical hemodynamics, adversely influence surgical outcome, and delay patient recovery. Recent reports suggest that reduced chest drainage increases mortality and is associated with longer stays in the intensive care unit, prolonged reliance on mechanical ventilation, and other adverse outcomes. Obstructions in drain catheters can further directly impact patient recovery after surgery. In serious cases, a surgeon may have to remove clots around the heart to prevent pericardial tamponade and resulting cardiogenic shock. This suggests that postoperative bleeding and resulting detrimental effects are possibly caused by inefficient clearing of blood due to occluded chest-tubes.

Traditionally, chest tube management employed to clear occlusions are improvised mechanical methods such as “milking” and tapping the portion of the tube external to the patient to dislodge clots and maintain patency of the chest tube. Another more controversial method of clearing blockages is chest tube stripping, which can generate significant temporary negative intrathoracic pressure and injure the tissue surrounding the drain. The milking of chest tubes can also inadvertently push clots and other material back into the intrathoracic portion of the drain tube that may occlude the tube eyelets on the internal portion of the chest tube. It has also been shown that the degree of occlusion cannot always be determined just by inspecting the tubes. The intrathoracic portion of the chest tube can be occluded even when the extra thoracic portion appears clear.

Currently, safe, reliable, methods to prevent or inhibit chest-tube blockages do not exist. The use of heparin coatings, electroactive polymers embedded in the chest tube that alternately expand and contract, variable diameter tubes, and tubes having local anesthetics do not adequately affect internal blockages or do not have a prolonged effect on preventing blockages.

Reduction of clot accumulation and other solidified body matter within the chest tubes and other surgical drains and complications associated therewith require more efficient systems, devices and methods to control and/or prevent clot formation on the inner lumen of chest tubes, drainage catheters, and other similar medical devices. There is a need for a drainage system, for chest and other body cavities, which reduces or prevents occlusions and can be reliably implemented, particularly after heart and lung surgery. Ideally, such a drainage system will operate continuously in the postoperative patient without requiring intervention from the healthcare providers.

### BACKGROUND OF INVENTION

The subject invention provides devices and methods that address the problem of blockages by occlusions formed in chest surgical drains and other body cavity surgical drains. Surgical drains are used to carry liquids, gases, solids, semi-solids, and other body matter and debris out of a body cavity. When this body matter adheres to the central lumen of the surgical drain tube, occlusions can form that inhibit further movement of body matter through the surgical drain tube and promotes the formation of additional occlusions. The drainage system of the subject invention can facilitate the removal of occlusions to maintain normal flow and movement of body matter through the surgical drain tube. The drainage system of the subject invention can operate without impeding flow within the surgical drain tube and, thus, does not promote the formation of occlusions as a usual result of reduced flow causing adherence of body matter and other debris to the interior wall surface of the surgical drain tube.

In one embodiment, the drainage system comprises a mechanized surgical drain with a carrier mechanism in the central lumen of the surgical drain tube that moves occlusion material through the surgical drain tube, and a drive unit to operate the carrier mechanism. The mechanized surgical drain can be placed within the body cavity, similarly to, for example, a thoracic surgical drain, and can be used in combination with a vacuum force to facilitate the movement of occlusion material through the drainage tube, placed within the body, to a suction port (outlet) at the proximal end. The suction port can be connected to collection hose that ultimately leads to a collection chamber for receiving and at least temporarily storing the secretions and other body material.

The carrier mechanism in the surgical drain tube lumen comprises a structure that can be rotated or turned within the surgical drain tube in a fashion that draws or pulls occlusion material along the length of the surgical drain tube. This can include, but is not limited to, forms such a coils, screws, springs, vanes, paddles, augers, similar rotatable carrier mechanisms, and combinations thereof.

The rotation of the carrier mechanism within the surgical drain tube can be achieved with a drive unit to which the carrier mechanism is operably attached. The drive unit can be located at a proximal end of the surgical drain tube, outside the body of the patient, and close to the incision site. Axial rotational movement of the carrier mechanism by a drive mechanism, in conjunction with the vacuum force, can pull, draw, carry, drag, or otherwise move or transport liquids, gases, solids, and semi-solids, as well as other blockage material through the surgical drain tube.

A drive mechanism and the carrier mechanism can be operably attached, so that the drive mechanism rotates the carrier mechanism in the lumen of the surgical drain tube. In one embodiment, the drive mechanism has a motor shaft to which the carrier mechanism is operably attached. In a further embodiment, the drive mechanism is a gearmotor that can rotate a motor shaft, to which the carrier mechanism can be operably attached. In a particular embodiment, the drive mechanism is a micro gearmotor. In a further embodiment, the gearmotor is operably connected to and axially rotates the carrier mechanism. Advantageously, the embodiments of the subject invention provide for an arrangement of the gearmotor in-line with the carrier mechanism, which allows for the carrier mechanism to be relatively short and less prone to kinking, binding, or entangling within the lumen of the surgical drain tube. In a specific embodiment, the carrier mechanism is located at least partially in the portion of the catheter placed in vivo. Certain embodiments of the of the subject invention utilize a gearmotor that includes a reduction gear box that enables the carrier mechanism to be rotated at relatively low speeds.

The drive unit employed with embodiments of the subject invention can be at least partially enclosed within a housing that can further insulate against noise and vibration from the gear motor. The drainage system, which includes the mechanized surgical drain, can be operably connected to a collection hose for draining body matter into a collection container. Certain embodiments employ a manifold with conduits to which the surgical drain tube, drive mechanism, and collection hose can be attached. A manifold can facilitate the movement of body matter through the surgical drain tube, through the manifold, and into collection hose that leads to a collection container. The manifold can also aid in maintaining a vacuum throughout the drainage system. Certain embodiments of a manifold can also include an injection flush port for removing occlusions that may develop within the manifold.

Patients having in vivo placed surgical drains often require one or more imaging procedures to monitor various aspects of their condition. A further advantage of the drainage system of the subject invention is that the materials utilized allow for the mechanized surgical drain to remain in vivo during imaging procedures. For example, the mechanized surgical drain can comprise a plastic or polymer, such as a fluorinated polymer, including polytetrafluoroethylene (PTFE), or a polyolefin such as polypropylene. One or more components can also comprise a transparent material that allows for visually monitoring the movement of body matter or other material through the drainage system. The surgical drain tube can have a longitudinal length that is flexible or semi-flexible. The surgical drain tube can also have a portion of the longitudinal length that is not flexible or semi-flexible. By way of a non-limiting example, some portion of the surgical drain tube that includes the proximal end can be rigid or semi-rigid. The rigid or semi-rigid portion can, but is not required, to include the suction outlet.

The drainage system of the subject invention successfully addresses the above described disadvantages associated with the previously known drainage tube devices and methods, and provides certain attributes and advantages, which have not been realized by these known devices. Advantageously, the drainage system of the subject invention can be readily incorporated into existing procedures and used with devices currently used in medical facilities, thereby minimizing training requirements. Beneficially, a drainage system of the subject invention improves removal of body matter from a body cavity by continuously removing occlusions in the surgical drain tube and consequently reducing the requirement of monitoring and intervention required by currently used drainage tubes. The compact size, lightweight, independent power supply (e.g., battery) provide a self-contained, continuously operating drainage system that allows patients to more easily ambulate and move during the recovery process while maintaining drain patency.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention.

FIG. 1A illustrates an embodiment of a drainage system, according to the subject invention. Shown here is a mechanized surgical drain with a tether arranged in a through-hole in a slant-cut distal end of the surgical drain tube.

FIG. 1B illustrates an exploded view of an embodiment of a drainage system, according to the subject invention.

FIG. 1C illustrates an exploded view of an embodiment of a drainage system, according to the subject invention. In this embodiment, a non-conductive coupler is arranged on the motor shaft.

FIG. 1D is a plan view of the embodiment of the drainage system, shown in FIG. 1C. In this view, the carrier mechanism has been truncated for clarity.

FIG. 1E shows an embodiment of a drain tube, according to the subject invention, to which a collection hose is directly attached over the suction port.

FIG. 2A illustrates an embodiment of a drainage system, according to the subject invention. In the embodiment shown, the mechanized surgical drain is implanted through an incision and into a body cavity and is operably connected to a vacuum collection hose leading to a collection chamber.

FIG. 2B illustrates embodiments of retainers, according the subject invention. Specifically shown are a clamp retainer and cuff retainer. A clamp retainer can be attached around a mechanized catheter after implantation. A cuff retainer can be place on or around the mechanized surgical drain in advance of implantation.

FIGS. 3A, 3B, and 3C illustrate embodiments of housings and manifolds that can be utilized with embodiments of the subject invention. The manifold embodiment shown in FIG. 3A has an outflow conduit in which a collection hose or medical fluid connector can be inserted. The embodiment in FIG. 3B has an exit port to which a collection hose or medical fluid connector can be attached. FIG. 3C shows a particular embodiment whether the outflow conduit configured as a fluid connector to which a collection hose can be connected. The embodiment in FIG. 3C also shows a manifold with an injection flush port that can be used, if necessary, to remove or advance occlusions that may form within the manifold.

FIGS. 4A and 4B illustrate portions of embodiments of a mechanized surgical drain, according to the subject invention. FIG. 4A illustrates an embodiment having bristles on the carrier mechanism. FIG. 4B illustrates an embodiment with inlets around the distal end through which body matter enters the mechanized surgical drain. Also shown is the common axes of the surgical drain tube and the carrier mechanism.

FIGS. 5A, 5B, and 5C illustrate devices and one method for placing and securing a mechanized surgical drain in vivo using a tether and a retainer, respectively. FIG. 5A shows one embodiment of a mechanized surgical drain in vivo and methods by which the mechanized surgical drain can be secured using two different embodiments of a retainer. FIGS. 5B and 5C show alternative embodiments of a tether connected to the mechanized surgical drain with a plug (FIG. 5B) and a tether that can be threaded through one or more through holes and one method for removal of the tether from the mechanized surgical drain after using the tether to pull the distal end through a surgical incision into a body cavity.

### DETAILED DISCLOSURE

The subject invention pertains to a drainage system advantageous for medical use. More specifically, the subject invention provides a mechanized surgical drain for use in removing body matter from a patient. The mechanized surgical drain of the drainage system of the subject invention can inhibit blockages by, advantageously, moving occlusion material through the length of the mechanized surgical drain for eventual removal to a collection container. Advantageously, embodiments of a mechanized surgical drain of the subject invention can be self-contained, having an on-board power source and operational features and that are sufficient compact to facilitate patient ambulation.

The subject invention is particularly useful in the field of surgical drains and catheters. In particular, embodiments of the subject invention are advantageous as chest tubes for draining fluids, and other body materials from body cavities, such as, for example, the pericardial and pleural cavities. Because the surgical drain tube can be relatively short, embodiments of the subject invention can provide surgical drain tubes of various diameters, making them useful for a variety of purposes and specifically beneficial in pediatric applications where small bore drain tubes are often used and are more prone to occluding. Thus, while the subject application describes, and many of the terms herein relate to or describe use as chest tubes, other uses and modifications apparent to a person with skill in the art and having benefit of the subject disclosure are contemplated to be within the scope of the present invention.

In the description that follows, a number of terms are utilized. In order to provide a clear and consistent understanding of the specification and claims, including the scope to be given such terms, the following definitions are provided.

The term “body cavity,” as used herein, is merely for literary convenience. Any fluid-filled space in a multicellular organism to which the embodiments of the subject invention can be applied and are useful is considered to be encompassed by this term.

Likewise, the term “body matter” is also used herein for literary convenience. The term refers to any liquid, gas, solid, or semi-solid substance or other material, biological or otherwise, that is required or desired to be removed from the body.

The terms “blockage,” “blockage material,” or “occlusion” refers to body matter and other material that inhibits the flow or movement through a surgical drain tube.

Furthermore, the terms “about” or “approximately,” as used herein, are defined as at least close to a given value or either end of a range as is necessary to cover manufacturing variances, equipment tolerances, and normal variances in material, as understood by those skilled in the art.

Also, as used herein, and unless otherwise specifically stated, the terms “operable communication,” “operable connection,” “operably connected,” “operable attached,” “cooperatively engaged” and grammatical variations thereof mean that the particular elements are connected in such a way that they cooperate to achieve their intended function or functions. The “connection” or “engagement” may be direct, or indirect, physical or remote

As used herein, the terms “longitudinally” or “longitudinal length” refer to the longitudinal measurement or the longitudinal distance along the long axis. For example, the longitude or longitudinal length of a mechanized drain tube is the distance between the distal end placed in a patient and the proximal end that is arranged in an inflow conduit of a manifold.

Finally, reference is made throughout the application to the “proximal end” or “proximal direction,” “distal end,” and “distal direction.” As used herein, distal refers to that end or direction that is closest to, within, or nearest to a patient Conversely, proximal refers to that end or direction that is furthest from or away from a patient.

The present invention is more particularly described in the following examples that are intended to be illustrative only because numerous modifications and variations therein will be apparent to those skilled in the art. As used in the specification and in the claims, the singular for “a,” “an” and “the” include plural referents unless the context clearly dictates otherwise.

It is to be understood that the Figures and descriptions of embodiments of the present invention have been simplified to illustrate elements that are relevant for a clear understanding of the invention, while eliminating, for purposes of clarity, other elements that may be well known. Those of ordinary skill in the art will recognize that other elements, well-known in the art, may be desirable and/or required in order to implement the present invention.

Reference will be made to the attached Figures on which the same reference numerals are used throughout to indicate the same or similar components. With reference to the attached Figures, which show certain embodiments of the subject invention, it can be seen that a drainage system 50 embodiment of the subject invention comprises a mechanized surgical drain 100, also referred to herein as a surgical drain, with an internal carrier mechanism 110 having motion capabilities that can move body matter 6 through the lumen 105 from a distal end 2 to a proximal end 4 of the surgical drain. The carrier mechanism of the mechanized surgical drain can be operably connected to drive mechanism 300 that can be disposed in a housing 400. The drainage system can include a manifold 450 with conduits 453 of which one can have at least the carrier mechanism arranged therein for operable connection to the drive mechanism and another that directs body matter from the suction port in mechanized surgical drain 100 to a collection tube 9. Embodiments of a drainage system can also include a tether 500 at the distal end 2 to assist with in vivo placement by maneuvering the mechanized surgical drain through a surgical incision and into a body cavity and a surgical drain tube retainer 600 for attachment at or near the drain incision site 11 to retain the in vivo mechanized surgical drain in position. Each of these general components can have one or more sub-components, which will be discussed in detail below.

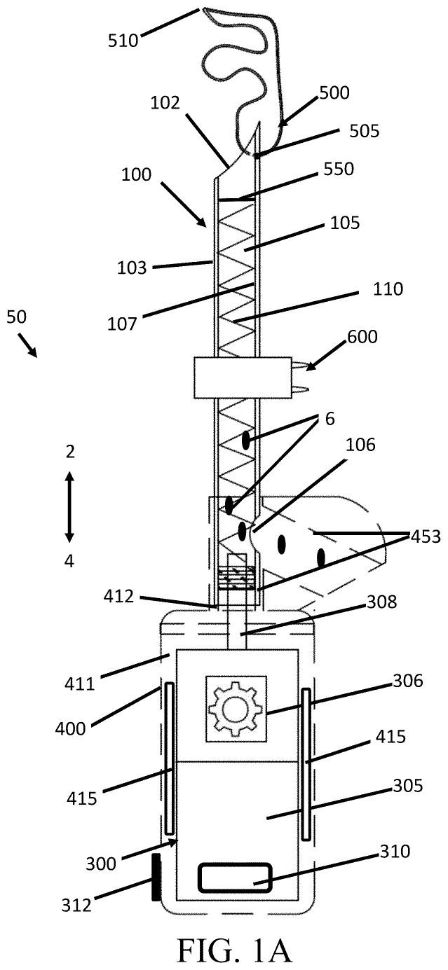

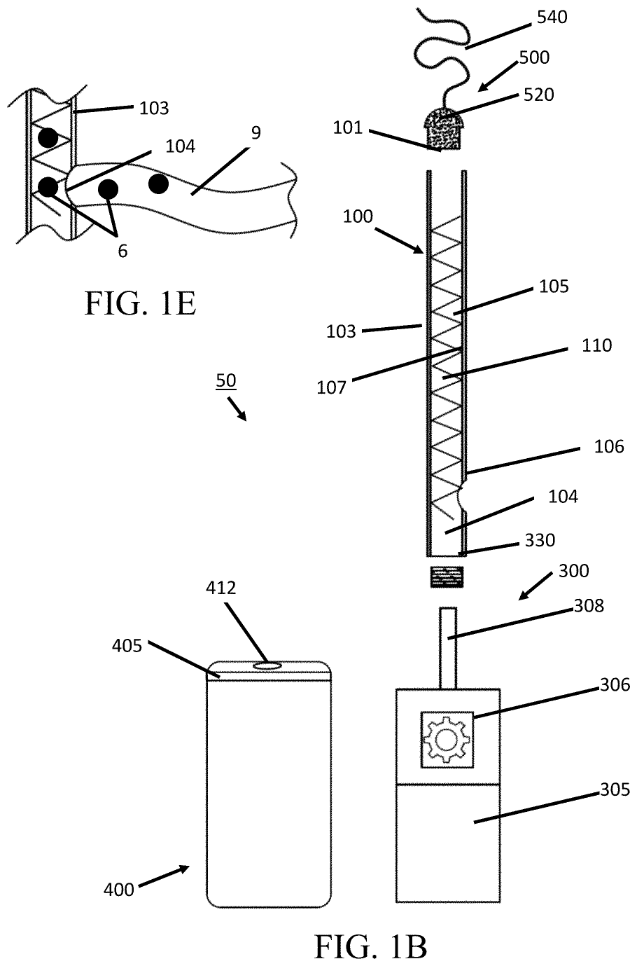

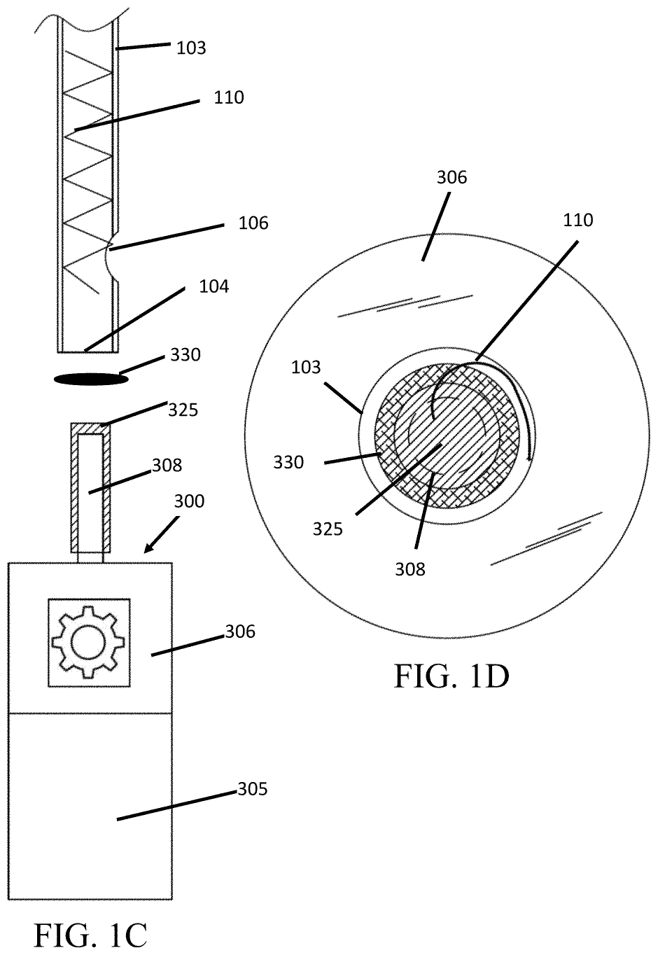

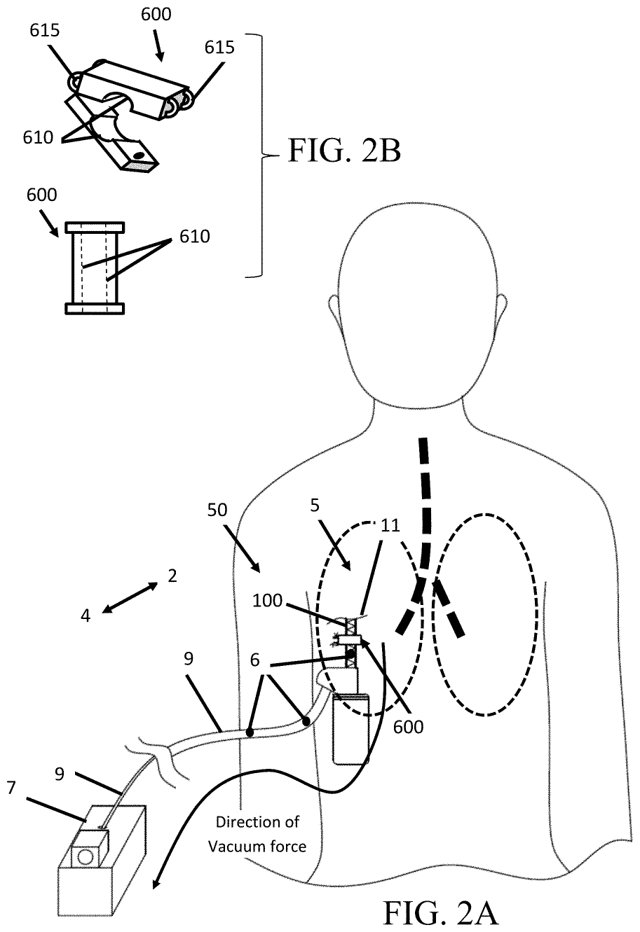

FIGS. 1A and 1B illustrate one embodiment of a drainage system 50 that includes a mechanized surgical drain 100 with an internal carrier mechanism 110 operably connected to a drive mechanism 300 that provides motion capabilities to the carrier mechanism. The surgical drain tube can be placed within a body cavity 5 to remove body matter 6 from a body cavity, which is then usually moved to a collection container 7. For example, FIG. 2A illustrates a mechanized surgical drain utilized as a chest tube placed in a pleural cavity to drain body matter that collects in the pleural cavity, which can occur, for example, after surgery or other trauma. The drainage system embodiments of the subject invention can advantageously be manufactured from one or more materials that can be sterilized and can also be utilized with imaging devices such as magnetic resonance imaging (MRI), computerized axial tomography (CAT), x-ray, and ultrasound. The drive mechanism can also be electrically isolated to inhibit damage or interference from other electronics, such as, for example, defibrillators (AED) or other electrical stimulation devices.

With reference to the Figures, one embodiment of the subject invention comprises a mechanized surgical drain 100, which can be placed in a body cavity, such as the chest cavity, of a patient. Suction or vacuum pressure can be applied to the drainage system to assist in pulling or drawing body matter through the mechanized surgical drain. Suction lines are commonly available in surgical suites and hospital rooms and can be connected to a drainage system 50 of the subject invention. Alternatively, collection containers 7, such as, for example, a Jackson-Pratt (JP) drain, often have a built-in pump to apply vacuum pressure to create a negative pressure in drainage catheter. Drainage system embodiment of the subject invention can be operably connected to such a collection container, so that the vacuum pressure forms a negative pressure within the mechanized surgical drain.

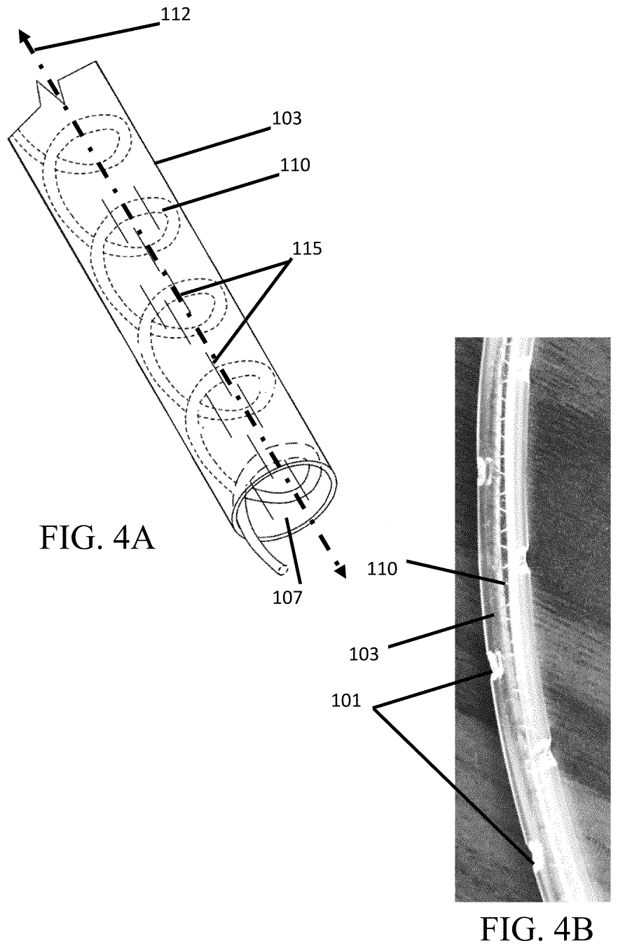

Embodiments of a mechanized surgical drain include a surgical drain tube 103 with an internal carrier mechanism 110. The surgical drain can have one or more inlets 101 through which body matter flows or moves from a body cavity 5 into the lumen 105 of the surgical drain. Inlets can be arranged in and around the surgical tube, particularly at the distal end placed in vivo. The distal end opening of the surgical drain can also be regarded as an inlet. FIG. 4B shows an example of a portion of a mechanized surgical drain of the subject invention that has a plurality of inlets.

To promote the movement or flow of body matter 6 through the inlets in the surgical drain and inhibit the formation of blockages, a carrier mechanism 110 can be disposed within the surgical drain tube central lumen 105. The carrier mechanism can have a diameter that allows it to be in at least partial or temporary contact with the interior surface 107 of the lumen 105 of the surgical drain tube and, in certain embodiments provides a scraping or rubbing action against the interior surface 107 to dislodge blockage material. Non-solid body material can move normally through the surgical drain, but can also be advanced through the surgical drain with the carrier mechanism. The carrier mechanism is particularly beneficial in advancing solid or semi-solid body matter through the surgical drain that may otherwise occlude or reduce flow in the surgical drain.

In one embodiment, the carrier mechanism 110 is coaxial with the lumen 105 and can turn, spin, twist, rotate, or otherwise move axially along the longitudinal length within the lumen 105. A carrier mechanism can include, but is not limited to, coils, vanes, augers, screws, combinations thereof or other devices or constructions capable of moving body matter through the lumen by movement thereof in the lumen. In one embodiment, the diameter of the carrier mechanism provides a scraping or rubbing action to inhibit and/or dislodge body matter adhered to the interior wall surface 107. The carrier mechanism can beneficially assist with the movement of body matter through the surgical drain tube 103 at lower suction pressures. For example, with a given diameter of surgical drain tube and appropriate suction pressure, body material can be moved through the lumen at a given flow rate. The axial rotation of the carrier mechanism in the lumen can increase the flow rate in that same surgical drain tube under the same suction pressure. If the suction pressure is reduced, the rotation of the carrier mechanism can assist in providing at least the same or similar flow rate as achieved at a higher suction without the carrier mechanism. In other words, the rotation of the carrier mechanism can supplement the flow rate produced by the suction pressure, which can reduce the suction pressure required to move body material through the drainage system 50. As discussed below, a drive mechanism can determine the rotation rate of the carrier mechanism.

Embodiments of a mechanized surgical drain 100 of the subject invention can be particularly beneficial as a chest or thoracic cavity surgical drain. When utilized as a chest tube, one embodiment of a surgical drain tube has a circular cross-section and an external diameter of between about 12 French and about 32 French, which can translate to an external diameter of between about 2 mm and about 12 mm. Typically, when a mechanized surgical drain 100 of the subject invention is utilized as a chest tube, the size of the surgical drain tube used can depend upon whether the patient is an adult or child. In one embodiment, the external diameter of a mechanized surgical drain, such as can be used in an adult patient, is between about 28 Fr and about 32 Fr or between about 9 mm and about 11 mm. In another embodiment, the external diameter of a mechanized surgical drain, such as can be used use in a child patient, is about 17 Fr to about 19 Fr or about 5 mm to about 7 mm. In yet another embodiment, the external diameter of a mechanized surgical drain, such as used in a newborn patient, is between about 12 Fr and about 14 Fr or between about 4 mm and about 5 mm.

A mechanized surgical drain 100 of the subject invention is not limited to chest tubes and can be utilized for any variety applications in which a body cavity needs to be drained. In the human body, this can include, but is not limited to, the thoracic cavity, abdominal cavity, cranial cavity, pelvic cavity, and any other body cavity or space in need of being drained. In specific embodiments, a mechanized surgical drain of the subject invention can have an external diameter of at least 1 mm, 5 mm, 10 mm, 15 mm, 20 mm, 25 mm, 30 mm, 35 mm, 40 mm, or 45 mm or an external diameter in a range between any two of the listed values.

The length necessary for a mechanized surgical drain can depend upon a variety of factors, such as the age of the patient, location of the cavity, size of the cavity, position in the cavity, patient positioning during the drainage procedure, and other factors known to those with skill in the art. A typical drainage catheter extends from the patient body cavity to a collection chamber. Embodiments of a mechanized surgical drain extend from the patient body cavity to a drive mechanism. Thus, embodiments of a mechanized surgical drain can be of any suitable length, between the distal end 2 positioned within the patient and the proximal end 4 where the mechanized surgical drain connects to the drive mechanism 300. Advantageously, the embodiments of the subject invention allow for a mechanized surgical drain 100 to be of relatively small diameter, short, and at least shorter than a standard drainage catheter, which inhibits disruptions to the operation of the carrier mechanism 110 and can be more comfortable for the patient. In one embodiment, a mechanized surgical drain can be at least 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, or 100 cm or a length in a range between any two of the listed values. In a particular embodiment, the length of a mechanized surgical drain is between about 30 cm and about 60 cm. In a specific embodiment, the length of a mechanized surgical drain is about 45 cm.

While embodiments of the drainage system 50 of the subject invention can inhibit the formation of blockages in a surgical drain, nonetheless, it can still be advisable for medical staff to monitor the surgical drain for other reasons, such as, for example, changes in color of the body matter, blockages that may be too large to be passed through the lumen, decreased flow, increased flow, and other issues that may arise. In one embodiment, the mechanized surgical drain of the subject invention comprises a transparent or semi-transparent material through which the body matter can be observed. The material of a surgical drain tube can be for example, silicone, polyurethane, polyethylene, polyvinylchloride, polytetrafluoroethylene, nylon, or other materials or combinations thereof considered safe for use with medical devices.

In one embodiment, the carrier mechanism 110 in a mechanized surgical drain 100 is a spring, coil, or screw arranged within the longitudinal length of the lumen 105 of the surgical drain tube. In another embodiment, the carrier mechanism 110 in a mechanized surgical drain has at least a portion that extends from the surgical drain, such as, for example, at the proximal end. The carrier mechanism 110 and the surgical drain tube 103 can have coaxial axes 112, an example of which is shown in FIG. 4A. Axial rotation of the spring, coil, screw, or similar device that creates an auger- or screw-like action that can convey, carry, drive, push, or otherwise move material within the lumen. The carrier mechanism can have flights that when rotated move material in either direction along the length of the screw, depending on the direction of rotation. There are a variety of springs, coils, or screws that can be employed in a mechanized surgical drain of the subject invention. Ribbon screws, including leg- or post-type ribbon screws, could be used, as well as shaft-less screws. In one embodiment, the carrier mechanism 110 is a shaft-less screw. In a specific embodiment, the carrier mechanism is a pre-formed helical coil 120, such as shown, for example, in FIGS. 1, 4A, and 4B. With this embodiment, the helical coil is rotated in the surgical drain tube 103, in a direction that carries body matter 6 away from distal end 2, which is in vivo, towards the proximal end 4, which leads to suction port 106. The axial rotation aids in maintaining patency of the surgical drain tube by moving body matter through the mechanized surgical drain. The axial rotation of the spring or helical coil provides a screw-like or auger-like action that causes blockage material, occlusions, and other solidified or semi-solidified body matter to be carried along the lumen. The direction of the rotation can determine the direction that material is carried in the lumen.

In one embodiment, the screw or helical coil is flexible along the longitudinal length, so that it can conform when the surgical drain tube is curved, bent, or arranged in another non-linear shape to facilitate in vivo placement. A screw or helical coil 120 of the subject invention can have any one or more of a variety of cross-section shapes including, but not limited to, circular, square, triangular, rectangular, oval, other polygonal shapes, and combinations thereof.

There can also be agitators 115 on the carrier mechanism, such as, for example, bristles, vanes, paddles, teeth, cut-outs, or other structures, which can serve advance body matter through the carrier mechanism, such as by mixing or agitating body matter and/or to scraping or rubbing the interior wall surface 107 of the surgical drain tube. FIG. 4A illustrates an embodiment of carrier mechanism in the form of a helical coil that has bristle attachments arranged to scrape or rub the interior wall surface 107 of the surgical drain tube 103 as the carrier mechanism 110.

A carrier mechanism, such as, for example, a helical coil, can comprise one or more of a variety of materials, such as, for example, metals, alloys, plastics, ceramics, polymers, rubber, nylon, combinations thereof or any other suitable material, determined by a person of skill in the art. Preferably, the material of the carrier mechanism has sufficient tensile strength that rotation of one end can translate along the longitudinal length, thereby rotating the longitudinal length of the carrier mechanism, but maintains sufficient longitudinal flexibility to conform or adapt to the curvatures, turns, bends and other non-linear formations of the outer surgical drain tube 103. Ideally, the one or more materials have a flexibility that allows the mechanized surgical drain to be conformable or bendable for placement in the patient and still facilitate rotation in the surgical drain tube of the carrier mechanism 100. It can also be beneficial for the material to be capable of withstanding sterilization procedures.

In a further embodiment, the carrier mechanism 110 can have a non-stick exterior surface that inhibits adherence of body matter passing through the surgical drain. For example, the carrier mechanism can be coated with one or more fluoropolymers, such as polytetrafluoroethylene or other non-stick materials with similar properties such as a polyolefin, including polypropylene, polyethylene, or combinations thereof. The exterior surface of the carrier mechanism could also be coated with drugs or substances that have anti-thrombogenic properties. Alternatively, the carrier mechanism can have a surface treatment that gives a polished finish that imparts anti-thrombogenic properties. One example of such surface treatment that can be used with a metallic carrier mechanism of the subject invention is magnetoelectropolishing (MEP). Such coatings and/or treatments can also facilitate rotation of the helical coil in the lumen by reducing stiction between the helical coil and internal wall surface 107 of the lumen 105.

The dimensions of a carrier mechanism can depend upon a variety of factors, including, but not limited to, the diameter of the surgical drain tube, the material utilized, the rate of rotation, the amount of suction pressure 8 applied to the system, and other factors known to those with skill in the art. In one embodiment, the diameter of a carrier mechanism, such as helical coil, and any attachments that may be thereon allow it to be inserted into the lumen 105 of a surgical drain tube 103. In another embodiment, the carrier mechanism can make at least intermittent contact with the interior wall surface 107, especially when moving or rotating.

In another embodiment, the carrier mechanism is about the same length as the surgical drain tube that will be employed in the patient cavity. In an alternative embodiment, the carrier mechanism is shorter than the length of the surgical drain tube that will be employed in the patient. For example, the length of the carrier mechanism can be less than the length of the surgical drain tube by about 10%, about 15%, about 20%, about 25%, about 30%, about 35%, about 40%, about 45%, or about 50% or a length in a range between any two of the listed values.

The rate of twist or number of full turns in a given length of a carrier mechanism, such as, for example, a helical coil or spring, can also vary. The greater the number of turns the more scraping action on the internal wall surface 107, but increased turns can also increase stiffness of the carrier mechanism and possibly impede rotation in the surgical drain. In one embodiment, a carrier mechanism has a rate of twist of between about 1 turn per inch and about 10 turns per inch. In a particular embodiment, a carrier mechanism has a rate of twist of between about 2 turns per inch and about 8 turns per inch. In a particular embodiment, a carrier mechanism has about 6 turns per inch.

The effectiveness of the carrier mechanism 110 in the surgical drain tube 103 is at least in part due to the axial rotation in the lumen 105. Rotation can cause the carrier mechanism to scrape, rub, or otherwise at least partially contact the interior wall surface 107 to dislodge body matter that has attached thereto or inhibit the attachment of body matter. A carrier mechanism in the form of a helical coil can be particularly advantageous. A helical coil can make regular, repeated contact with the interior wall surface along the length of the coil. Thus, a helical coil that extends through some or all of the length of the surgical drain tube, as shown, for example in FIGS. 1, 4A and 4B, can make at least intermittent contact the interior wall surface along that length. Additionally, the helical coil can operate similarly to an auger, so that, as it turns, body matter causing blockage in the surgical drain is dislodged and the coil rotation can act to move the blockage material towards the proximal end 4 for removal from the surgical drain.

Rotation of the carrier mechanism 110 can be achieved by rotating one end in a circular manner, so that the tension and tensile strength of the material of the carrier mechanism causes the rotation to be transferred along the longitudinal length so as to rotate the full length of the carrier mechanism. In one embodiment, the proximal end 4 of the carrier mechanism is attached to an apparatus or device that can provide axial rotational mechanical energy of sufficient force or torque to rotate the length of the carrier mechanism.

In many situations, it is beneficial for a patient to be ambulatory as soon as possible and as much as possible after surgery, an injury, or other physical trauma. If recovery requires in vivo placement of a surgical drain tube, such ambulation can include sitting up in bed or in a chair, standing, and walking. Advantageously, embodiments of a mechanized surgical drain tube 100 of the subject invention can be small, lightweight, and have an independent power source, such as, for example, a battery operation with a control switch 312, providing a self-contained drainage system 50 that can facilitates patient ambulation. The mechanized surgical drain 100 embodiments of the subject invention are advantageous in reducing blockages, which can allow smaller diameter surgical drains to be placed. A further advantage is the use of a carrier mechanism 110 within the surgical drain that can be rotated with a relatively simple, small drive mechanism 300. The compact size of embodiments of a drainage system 50 of the subject invention allows for closer placement to the patient and the incision site 11. This proximity to the patent and incision can also beneficially reduce the overall length of the carrier mechanism.

Gear motors are used for a plethora of mechanical and electrical applications. They can have significant power and torque capabilities and can be scaled to practically any size. A gear motor can include at least an electric motor 305 (AC or DC power source 310) and a reduction gear box 306 that operate to axially rotate a motor shaft 308 at a predetermined, often adjustable, speed (rpms). Gear motors typically have one of two types of gear arrangements: spur gear and planetary gear that regulate the rotation of a motor shaft. Embodiments of the subject invention can utilize either type of gear motor as a drive mechanism 300 for rotation of the carrier mechanism 110. In a specific embodiment, the drive mechanism is a micro gear motor. FIGS. 1A and 1B illustrate non-limiting examples of a micro gear motor employed with embodiments of the subject invention.

Surgical drains are usually secured within the incision with sutures. It is imperative that the surgical drain not be pulled or pushed after it has been properly positioned within the patient and secured with the sutures. Ideally, micro gear motors that can be utilized with embodiments of the subject invention are light-weight and small in size. A micro gear motor employed with embodiments of the subject invention can weigh between approximately 0.3 oz. and approximately 1.0 oz. Heavier or more robust micro gear motors are known in the art and can be used for larger mechanized surgical drain embodiments of the subject invention.

The motor shaft 308 of a micro gear motor can extend from or through a reduction gear box 306, as shown in FIGS. 1A and 1B. In one embodiment, at least part of the motor shaft is inserted into an outlet end 104 of the drain tube 103 to be fixedly connected to the proximal end 4 of the carrier mechanism 110. The carrier mechanism can be attached or fixedly connected to the motor shaft by any devices and techniques known in the art, such as, for example, welding, adhesives, or mechanical coupling. In one embodiment, the carrier mechanism is fixedly attached to the motor shaft so that axial rotation of the motor shaft simultaneously rotates the carrier mechanism. The motor shaft can rotate the carrier mechanism in either an axial clockwise or axial counterclockwise direction, whichever causes body matter to be drawn or pulled towards the motor shaft. This configuration of the motor shaft and carrier mechanism can position the motor shaft in-line with the carrier mechanism, as shown, for example, in FIGS. 1A-1C. The axial rotation of the carrier mechanism can be in-line with or approximately in-line with the axial rotation of the motor shaft. The in-line arrangement can direct the rotational power of the gear motor directly to the carrier mechanism, which can be efficacious in inhibiting kinking, binding, entangling, or other discombobulations that can impede rotation and operation of the carrier mechanism.

The speed at which the gear motor turns the motor shaft 308 should be sufficient to efficiently move body matter through the surgical drain. In one embodiment, the gear motor turns at a rate of between approximately 1 rpm and approximately 60 rpm, between approximately 10 rpm and approximately 50 rpm, between approximately 20 rpm and approximately 40 rpm, between approximately 30 rpm and 35 rpm or at a speed in a range between any two of the listed values.

In one embodiment, at least the motor shaft 308 of the drive mechanism 300 comprises an electrically non-conductive material. In an alternative embodiment, an electrically non-conductive coupler 325 is arranged on the motor shaft. In a further alternative embodiment, the carrier mechanism 110 is fixedly attached to the non-conductive coupler, such that the carrier mechanism is attached to the motor shaft by the non-conductive coupler. FIG. 1C illustrates an example of a non-conductive coupler on a motor shaft. FIG. 1D illustrates how a non-conductive coupler can isolate the motor shaft from the carrier mechanism 110. A motor shaft of non-conductive material or a non-conductive coupler can inhibit conduction of electric current through the carrier mechanism to the drive mechanism 300. This can be beneficial in situations where a drainage system 50 of the subject invention is implanted in a patient that is subsequently treated with a defibrillator or other electrical stimulation. The non-conductive coupler can protect the drive mechanism and the patient. There are numerous commercially available non-conductive materials for motor shafts and couplers. It is within the skill of a person trained in the art to determine an appropriate non-conductive coupler material for use with an embodiment of a drainage system of the subject invention.

In one embodiment, the drive mechanism 300 can be disposed in a housing 400. In one embodiment, the housing has an attachment device for securing the housing to a patient or patient clothing. For example, the housing can include wings or flanges to which adhesive tape can be attached. For another example, the housing can include clips, pins, ties, snaps, or other devices for securing the housing near the patient. A person of skill in the art can determine any of a variety of attachment devices that can be used with a housing and such variations are within the scope of the subject invention.

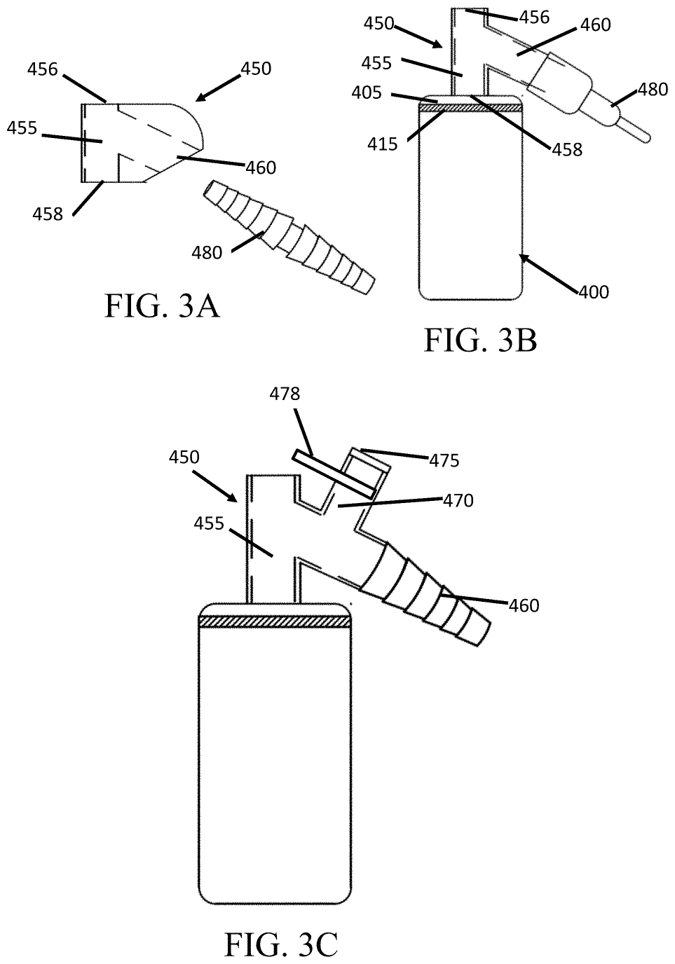

The housing can at least partially cover the drive mechanism 300. The housing can also provide a means by which the drive mechanism can be secured to or held in proximity to the patient. The housing can also inhibit noise and vibration from the drive mechanism. In a further embodiment, one or more insulators 415 can be used with the housing to muffle or reduce noise and vibration from the drive mechanism 300. By way of example, FIG. 3B illustrates a housing with cover 405 and a gasket therebetween. The gasket can comprise a material that performs as an insulator, thereby inhibiting vibrations and noise from the drive mechanism from being translated to the patient. The housing interior 411 can also include one or more insulators, such as shown in the example in FIG. 1A. Furthermore, the housing can comprise one or more materials that allow the housing to be an insulator.

As mentioned above, it can be beneficial for the drainage system 50 to be lightweight to reduce pulling on the sutures securing the mechanized surgical drain 100 in the patient. It can also be beneficial for the drainage system to be self-contained, such that the drive mechanism and power source. Preferably, the combined weight of the drive mechanism, on-board power source, e.g., battery, and housing is not more than a few ounces. In one embodiment, the combined weight of the drive mechanism 300, power source 310, and housing is between approximately 1 ounce and approximately 4 ounces, approximately 1 ounce and approximately 3 ounces, approximately 1 ounce and approximately 2 ounces, or a weight in a range between any two of the listed values.

Embodiments of a drainage system 50 of the subject invention are used to convey body matter 6 from a body cavity 5 to a collection tube 9 to be deposited in a collection container 7, as demonstrated in the example in FIG. 2A. Collection containers are known in the art and are commonly used in medical facilities and hospitals. To promote the movement of body matter 6 into a collection container, a vacuum or suction line is usually interconnected with the collection container. This can create a negative pressure in the collection hose that operates to pull material through a collection hose 9 that leads to the collection container. The vacuum pressure applied to the collection hose is usually between about 5 cm H₂O to about 25 cm H₂O.

Embodiments of a drainage system 50 of the subject invention can be configured to utilize the vacuum pressure. In one embodiment, at least a portion of the motor shaft 308 is inserted into an outlet 104 at the proximal end 4 of the mechanized surgical drain 100. The carrier mechanism 110 is fixedly attached to that portion of the motor shaft within the surgical drain tube, as is shown, for example, in FIGS. 1A and 1B. Rotation of the carrier mechanism can move body matter 6 towards the proximal end 4 and the motor shaft. In a further embodiment, the mechanized surgical drain has a suction port 106 near the proximal end of the mechanized surgical drain, an example of which is shown in FIGS. 1A, 1B and 1C. The suction port can provide a passageway between the mechanized surgical drain 100 and the collection hose 9 from the collection container 7. FIG. 2A illustrates an example of body matter 6 draining from a body cavity 5 through the inlets 101 in the drain tube 103 and into the mechanized surgical drain. The advantageous rotation of the carrier mechanism 110, in addition to the vacuum force generated through the drainage system 50 creates a negative pressure in the mechanized surgical drain 103 that encourages body matter to move or flow into the inlets. The body matter can be pulled towards the suction port 106 where it exits the mechanized surgical drain into a collection hose 9 and ultimately to a collection container, as shown in FIG. 2A.

The flow of body matter 6 towards the suction port 106 can also direct the body matter towards the motor shaft 308, which can be at least partially inserted into the proximal end of the mechanized surgical drain, as described above. To isolate the drive mechanism 300 from the body matter, a seal 330 can be arranged proximal to the suction port 106 to inhibit body matter from coming into contact with the drive mechanism. In one embodiment, the seal is arranged between the motor shaft 308 and the drain tube 103 to prevent body matter from flowing towards the drive mechanism. As described above, a non-conductive coupler 325 can be disposed on the motor shaft. In one embodiment, the seal is arranged on or around the non-conductive coupler. In a further embodiment, the seal is arranged on the motor shaft or the non-conductive coupler and extends annularly around the motor shaft and in contact with the drain tube. In a particular embodiment, the non-conductive coupler is configured as a seal. For example, a seal can be a shoulder or protrusion on the motor shaft or the non-conductive coupler. In another example, the seal can be an 0-ring or ring gasket arranged on the motor shaft or the non-conductive coupler. FIGS. 1A through 1D illustrate a non-limiting example of a seal 330 arranged between a motor shaft and a drain tube to inhibit body matter from flowing proximal to the seal. In a further embodiment, the seal not only inhibits fluids and body matter from contacting the drive mechanism, but also assists in maintaining the vacuum pressure through the system. A person with skill in the art can determine an appropriate seal for protecting the drive mechanism from body matter and other fluids. Variations in the type or configuration of a seal that provide the same functionality, in substantially the way as described herein, with substantially the same desired results, are within the scope of this invention.

Embodiments of a drainage system 50 of the subject invention can be operably connected to a collection hose 9 that empties into a collection container 7, as discussed above. More particularly, embodiments of a mechanized surgical drain 100 are operably connected to a collection hose via a suction port 106 through which body matter 6 flows. There are a variety of techniques and devices that can facilitate the operably connection between a drainage system 50 of the subject invention and a collection hose.

In one embodiment, a collection hose 9 is attached directly to the drain tube 103 over the suction port. Body matter can flow through the mechanized surgical drain and exit through the suction port directly into the collection tube. One example of this type of arrangement is shown in FIG. 1E. As mentioned above, at least a portion of the surgical drain tube 103 can be rigid or semi-rigid, including the suction port 106. In one embodiment, a collection hose 9 can be operably attached to the rigid or semi-rigid portion over or aligned with the suction port.

In another embodiment, the mechanized surgical drain 100 and a collection hose 9 are operably connected by a manifold 450. A manifold can include one or more conduits 453 that facilitate the flow of body matter 6 between the mechanized surgical drain and the collection hose. A manifold can be separate from or cooperatively engaged with or attached to a housing 400. Thus, embodiments of a drainage system 50 of the subject invention can include a housing, a manifold or both. FIG. 3A shows an embodiment of a manifold and FIG. 3B shows an embodiment of a manifold combined with an embodiment of a housing.

In one embodiment, at least a portion of the mechanized surgical drain, including the suction port 106, is arranged within an inflow conduit 455. In a further embodiment, the manifold has an outflow conduit 460 to which the collection hose is operably attached. In a particular embodiment, the outflow conduit connects with, intersects with or is otherwise continuous with the inflow conduit. In further embodiment, the drive mechanism is operably attached to the carrier mechanism within the portion of the mechanized surgical drain 100 arranged within the inflow conduit. Thus, the inflow conduit can also have a distal conduit opening 456 for receiving the mechanized surgical drain and a proximal conduit opening 458 for receiving the motor shaft 308. FIGS. 1A, 3A, and 3B illustrate non-limiting examples of manifolds having inflow and outflow conduits. The dimensions of an inflow conduit can be such that at least that portion of the proximal end of the mechanized surgical drain with the suction port can be arranged therein. The suction port can be positioned so that it is directed at or near to the outlet conduit so there is operable communication between the mechanized surgical drain and the outlet conduit, via the suction port. As shown in FIG. 1A, the dimensions of the inflow conduit can also encompass that portion of the surgical drain in which the motor shaft 308 is received. As mentioned above, the carrier mechanism 110 can extend from the surgical drain tube 103, as shown, by way of example, in FIG. 4A. In one embodiment, the carrier mechanism extends from the proximal end 4 of the surgical drain tube 103 to be operably attached to the motor shaft 308 within the inflow conduit 455. In an alternative embodiment, the carrier mechanism is not a long as the surgical drain tube, such that it terminates before the proximal end, such as shown, for example, in FIG. 1B. The motor shaft can be inserted into the proximal end of the surgical drain tube to connect with the proximal end of the carrier mechanism.

The outflow conduit 460 of a manifold can operably connect to the collection hose 9. Such operably connection can be achieved by directly inserting the collection hose into the outflow conduit, which is not shown in the Figures, but would be understood by a person of skill in the art. Alternatively, any of a variety of medical fluid connectors 480 can be used to couple the collection hose to the outflow conduit. FIGS. 3A and 3B illustrate examples of medical fluid connectors that can facilitate coupling of the collection hose to the outflow conduit. Other medical fluid connectors could also be used. In one embodiment, a fluid connector is integrated with or is formed as part of the outflow conduit 460. This embodiment permits a collection hose to be directly attached to the outflow conduit. FIG. 3C illustrates a non-limited embodiment of an outflow conduit shaped as a fluid connector on which a collection hose can be connected.

Once body matter leaves the surgical drain the vacuum force continues to draw the body matter through the outflow conduit 460 and into the collection hose 9. A typical collection hose has a larger diameter than the surgical drain, thus, it is less likely, but not impossible, for occlusions to form in the collection hose. In the event that occlusions do form in the collection hose, or the outflow conduit, it would be preferable to flush the outflow conduit and collection hose rather employ techniques such as stripping, milking or tapping to release occlusion material.

One embodiment of a manifold 450 of the subject invention comprises an injection flush port conduit 470. In a further embodiment, the injection flush port conduit is integral with the outflow conduit 460. An injection flush port conduit can have a one-way injection port 475 that allows a fluid, such as saline, sterile water, or other substance to be injected or forced into the injection flush port conduit so that it passes into the outflow conduit to dislodge occlusions in the outflow conduit and/or the collection hose. The vacuum force applied to the drainage system can encourage the fluid to pass into the outflow conduit. One-way injection ports are known in the art and any of a variety can be incorporated with an injection flush port conduit. FIG. 3C shows one embodiment of a manifold with an injection flush port conduit and injection flush port arranged thereon. Injection flush ports can include supplemental components 478, such as, for example, stop cock valve to close one or more other conduits in the manifold.

Placement of a surgical drain in a body cavity is typically done during a surgical procedure where the body cavity is accessible, such as by a thoracotomy. The placement procedure usually requires making an incision through the cavity wall. Typically, the proximal end 4 is passed or forced through the incision so that end is outside the body. The distal end is then positioned in the body cavity to maximize fluid flow. The use of a drive mechanism 300 at the proximal end 4 of a mechanized surgical drain tube 103 of the subject invention can necessitate a slightly different approach. For example, in a thoracotomy an incision is made through the chest wall and the distal end 2 of the mechanized surgical drain 100 can be introduced through the incision from outside the body and into the pleural cavity. Thus, the proximal end 4 of the mechanized surgical drain does not have to be passed through the incision. While the chest or other cavity is open, the surgeon can manually position the distal end of the mechanized surgical drain to maximize drainage of body matter out of the cavity. The cavity can be closed and the surgical drain held in place in the incision by strategically placed and tightened sutures and/or using a biologically compatible adhesive around the incision and surgical drain.

The flexibility of a standard surgical drain tube 103 often necessitates it be pushed through the incision with a forceps, finger, or other implement, which necessitates that the incision 11 be large enough to accommodate both the surgical drain tube and the implement. Deformation of the surgical drain tube during placement is often not an issue, as it will resume normal shape after positioning. Embodiments of a mechanized surgical drain tube 103 of the subject invention can also be manipulated by hand or with instruments to position them within the patient. It can be preferable not to force a mechanized surgical drain 100 through the incision. With mechanized surgical drain tube embodiments of the subject invention, the process of grasping and forcing the distal end 2 through an incision 11 and into the body cavity 5 can potentially damage the carrier mechanism 110. This is not to say that the mechanized surgical drain cannot be manipulated, but rather care should be exercised.

One embodiment of the subject invention includes a tether 500 removably connected at or about the distal end 2 of the mechanized surgical drain 100. A tether, which can be easily manipulated through an incision, can be used to pull the distal end of the mechanized surgical drain through an incision. This can allow for a smaller incision to be made and does not require that the mechanized surgical drain be squeezed or grasped, which reduces the likelihood of damage or operational interference, especially to the carrier mechanism 110. The tether provides an apparatus for manipulating the mechanized surgical drain. In one embodiment, the tether is narrow and flexible. For example, the tether can be a string, line, tape, cable, wire, or similar type of device and made of any biocompatible, sterilizable material.

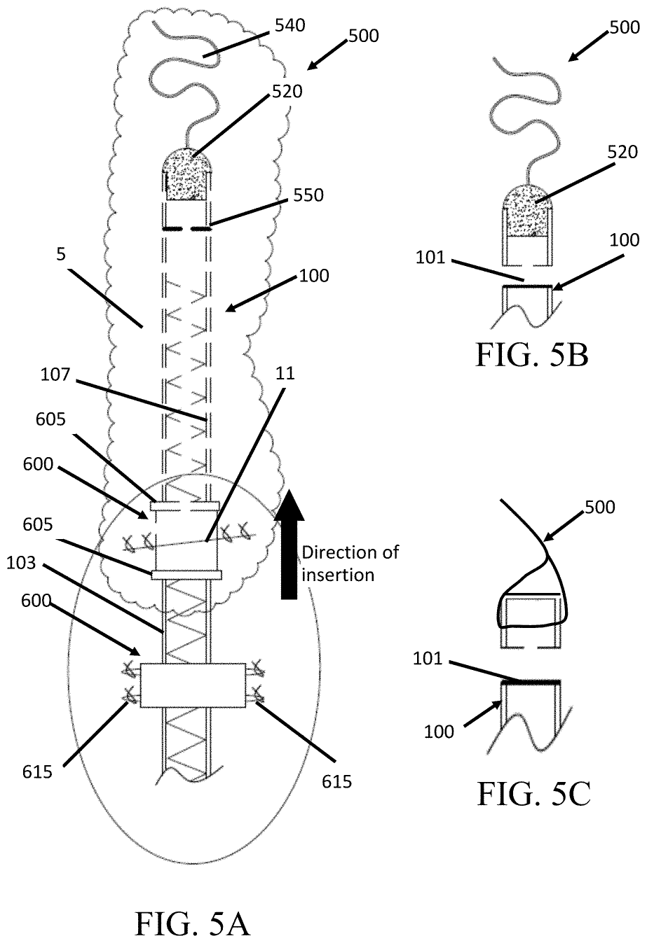

In one embodiment, a tether includes a plug portion 520, which inserts into, goes around, or goes over the distal end 2 of the mechanized surgical drain and the elongate tether 500 is fixedly attached to the plug portion. FIGS. 1A, 1B and 5A illustrate examples of a tether attached to a plug portion. In an alternative embodiment, the tether is attached directly to the mechanized surgical drain, for example, the tether can be connected or attached at or about the distal end 2 of the mechanized surgical drain. FIG. 5C illustrates a non-limiting example of this embodiment.

Drainage catheters often have a slant-cut proximal end 4, which aids in inserting the proximal end through an incision to the outside of the body. Because embodiments of a surgical drain tube 103 of the subject invention are manipulated in the opposite direction, as shown in the example in FIG. 5A, i.e., through the outside of the incision 11 and into a body cavity 5, it can be more advantageous for the surgical drain tube 103 to have a slant-cut distal end 102. In one embodiment, the slant-cut distal end of a surgical drain tube has at least one through-hole 505 through which a tether 500 can pass. The free end 510 of the tether 500 can be introduced into the body cavity through an incision using an implement, such as forceps, to which the free end can be attached or that can hold or grasp the free end. The tether can be pushed into the body through an incision, can be used to further pull the mechanized drain tube through the incision.

FIG. 5A illustrates the distal end 2 of a mechanized surgical drain 100 that has been maneuvered through an incision 11 with the tether 500 and into a body cavity 5. After the distal end of the mechanized surgical drain is in the body cavity, the tether can be removed from the distal end of the mechanized surgical drain. In one embodiment, the tether is attached to a plug that is pulled out of the distal end of the mechanized surgical drain, leaving an opening at the distal end, such as shown, by way of example in FIG. 1B. In an alternative embodiment, the tether is cut, which is not shown, but would be understood by a person with skill in the art. In yet another alternative embodiment, the distal end of the surgical drain tube 103 is cut or severed and removed with the tether to leave an inlet 101 at the distal end. In a further embodiment, there can be an indicator mark 550, for example, as shown in FIG. 5A, to indicate where a cut should be made to remove the tether without damaging the carrier mechanism. Once the distal end 2 of a typical drain tube is in vivo it is secured with sutures that close and compress the incision around the drain tube. Sometimes the sutures are wrapped around the drain tube and the incision. The compression around the drain tube can cause constriction of the lumen. With a typical drain tube such constriction, if minimal, can still allow body material to flow through the incision area and into the rest of the drain tube.

With embodiments of a mechanized surgical drain 100 of the subject invention, it can be important that the tightening sutures not constrict the lumen 105 of the surgical drain tube 103, particularly if the carrier mechanism 110 is close to or in contact with the internal wall surface 107. Constriction of the lumen of a surgical drain tube can damage or at least inhibit movement or rotation of the carrier mechanism.

In one embodiment, a retainer 600 is attached around at least a portion of the mechanized surgical drain. The retainer can engage around the drain tube 103 of the mechanized surgical drain and can provide a surface, platform, or structures to which tightening sutures can be attached. Ideally, the retainer has sufficient rigidity that when engaged with the surgical drain and tightening sutures are used to hold the retainer in place near the incision, the retainer can inhibit the tightening sutures from constricting the lumen 103. In a further embodiment, the retainer is moveable or adjustable along the length of the mechanized surgical drain, allowing for proper placement and adjustment in the patient.

By way of example, a retainer 600 can be a cuff around the exterior of the mechanized surgical drain, such as shown in FIG. 2B (bottom) and FIG. 5C (top). Tightening sutures can be wrapped around the cuff to hold it in or near the incision. There can also be one or more ridges 605 that inhibit the tightening sutures from sliding off the cuff. In use, the cuff can have a surface 610 that forms a friction fit with the exterior of the mechanized surgical drain that is sufficient to hold the cuff in place on the exterior of the mechanized surgical drain, thereby also holding it in place in the incision. If necessary, the cuff is slidable along the length of the mechanized surgical drain for adjustment in the patient.

By way of another example, the retainer 600 can be a clamp that is place around the exterior of the mechanized surgical drain, such as shown in FIGS. 2B (top), 5A (bottom), and 5B. The clamp can have one or more openings 615 through which tightening sutures are passed to hold the clamp in place in or near the incision. In use the clamp can have surfaces 610 that form a friction fit when is closed over the mechanized surgical drain that is sufficient to hold the clamp in place. If adjustment is needed the clamp can be opened and reclosed after the surgical drain is adjusted, preferably without disturbing the tightening sutures.

Drainage catheters for removing body matter that can collect in body cavities are a necessity after certain types of surgery or injury. Unfortunately, they have a tendency to be occluded by the body matter, which can solidify or clot in the catheter. This requires monitoring by medical personnel. The procedures for dislodging blockage material are not always fully successful and can be dangerous to the patient. The mechanized surgical drain embodiments disclosed herein provides a significant improvement in drainage catheter technology. Mechanized surgical drain embodiments can inhibit blockages. Blockages that form in the mechanized surgical drain can be moved or carried through the surgical drain by the mechanized surgical drain. This can reduce the amount of monitoring required by medical personnel and provide a safer, more reliable drainage tube for patients.

Any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A drainage system, configured to remove body matter from a body cavity, comprising:
a mechanized surgical drain comprising,
a surgical drain tube comprising a lumen, a proximal outlet end, and a suction port distal to the proximal end, such that body matter moves through the lumen towards the outlet end to exit through the suction port,
a carrier mechanism within and coaxial with the lumen,
a drive mechanism operably attached at or about a proximal end of the carrier mechanism that provides motion capability to the carrier mechanism,
such that, when body matter enters the surgical drain tube, the motion capability of the carrier mechanism within the lumen moves the body matter towards the suction port, thereby maintaining patency of the surgical drain tube.

2. The drainage system according to claim 1, wherein the carrier mechanism comprises a flexible coil, spring, or screw.

3. The drainage system according to claim 2, further comprising a motor shaft operably connected to the drive mechanism that rotates the motor shaft, wherein the motor shaft is operably attached to the carrier mechanism to axially rotate the carrier mechanism within the lumen.

4. The drainage system according to claim 3, wherein the drive mechanism is a gear motor.

5. The drainage system according to claim 3, further comprising a seal proximal to the suction port to inhibit body matter from contacting the drive mechanism.

6. The drainage system according to claim 3, further comprising a manifold having an inflow conduit for receiving the proximal outlet end of the surgical drain, an outflow conduit that receives the body matter through the suction port, and an injection flush port conduit.

7. The drainage system according to claim 6, further comprising the outflow conduit configured as a connector for a fluid collector hose.

8. The drainage system according to claim 6, further comprising a housing, wherein the drive mechanism is at least partially contained within the housing.

9. The drainage system according to claim 8 further comprising a power source for the drive mechanism contained within the housing.

10. The drainage system according to claim 6, further comprising a non-conductive coupler on the motor shaft.

11. The drainage system according to claim 6, further comprising a tether operably attached to a distal end of the mechanized surgical drain.

12. The drainage system according to claim 11, further comprising a slant-cut distal end on the surgical drain tube to which the tether is operably attached.

13. The drainage system according to claim 11, further comprising a plug fixedly attached to a distal end of the mechanized surgical drain, to which the tether is operably attached.

14. The drainage system according to claim 6, further comprising a retainer for securing the mechanized surgical drain within an incision.

15. The drainage system according to claim 14, wherein the retainer is a cuff or clamp arranged on the mechanized surgical drain.

16. A method, for removing body matter from a body cavity, comprising:
obtaining a drainage system, according to claim 1;
inserting a distal end of the mechanized surgical drain into the body cavity through an incision;
securing the mechanized surgical drain in the incision to maintain position of the distal end within the body cavity;
applying a vacuum to the suction port; and
activating the drive mechanism to provide the motion capability to the carrier mechanism that moves the body matter towards the suction port.

17. The method according to claim 16, wherein the distal end of the mechanized surgical drain has slant-cut.

18. The method according to claim 16, further comprising a manifold having an inflow conduit in which the proximal outlet end of the mechanized surgical drain is disposed and an outflow conduit configured to receive the body matter from the suction port and the method further comprises applying the vacuum to the outflow conduit.

19. The method according to claim 18, further comprising a tether at the distal end of the mechanized surgical drain and the method further comprises utilizing the tether pull the distal end of the mechanized surgical drain through the incision and into the body cavity.

20. The method according to claim 19, further comprising removing the tether from the mechanized surgical drain.

21. The method according to claim 18, wherein the carrier mechanism comprises a coil, spring, or screw and activating the drive mechanism causes an axial rotation of the carrier mechanism within the lumen.

22. The method according to claim 18, further comprising securing the position of the mechanized surgical drain in the body cavity using a retainer.

23. A kit, for draining body matter from a body cavity, comprising:
a drainage system, according to claim 1, and
a retainer.

24. The kit according to claim 17, wherein the drainage system further comprises a manifold comprising an inflow conduit in which the proximal end of the mechanized surgical drain is disposed, including the suction port, and an outflow conduit continuous therewith and to which the medical fluid connector is operably connected, such that vacuum pressure is applied to the mechanized surgical drain through the suction port.
